(19) United States
(12) Patent Application Publication
(10) Pub. No.: US 2021/0241065 A1
(43) Pub. Date: Aug. 5, 2021

(54) Classifying Discipline-Specific Content Using a General-Content Brain-Response Model

(71) Applicant: Brainvivo Ltd., Tel Aviv (IL)

(72) Inventors: Yaniv Assaf, Tel Aviv (IL); Assaf Horowitz, Tel-Aviv (IL)

(21) Appl. No.: 17/148,607

(22) Filed: Jan. 14, 2021

Related U.S. Application Data
(63) Continuation-in-part of application No. 16/667,198, filed on Oct. 29, 2019.
(60) Provisional application No. 62/775,018, filed on Dec. 4, 2018.

(51) Int. Cl. G06N 3/00; G06K 9/64; G06K 9/62; G06F 3/01

(52) U.S. Cl. CPC G06N 3/004; G06K 9/64; G06F 3/015; G06K 9/6232; G06K 9/6215; G06K 9/628

(57) ABSTRACT

A content classification method includes receiving a set of content items from categories of a specific discipline, and extracting respective features from each content item. A labeling of the content items of the specific discipline is received, performed by human viewers, the labeling indicating a respective category assigned to the content item by the human viewers. A general-content brain-response model is uploaded, the model estimated using measurements of brains of humans presented with a general-content database defined using a set of features and includes a mapping between the set of features and a set of extracted brain activities. The model is applied to the extracted features, to calculate, using the labeling, a set of brain-responses for the specific discipline. Given a new content item associated with the discipline, a category of the discipline best matching the new content item is estimated, based on the model and the discipline-specific brain responses.

### CROSS-REFERENCE TO RELATED APPLICATIONS

The present application is a continuation-in-part of U.S. patent application Ser. No. 16/667,198, published as U.S. Patent Application Publication 2020/0170524, titled “Apparatus and method for utilizing a brain feature activity map database to characterize content,” whose disclosure is incorporated herein by reference.

### FIELD OF THE INVENTION

The present invention relates generally to Artificial intelligence (AI). More particularly, this invention is directed toward techniques for automated classification of discipline-specific content using a brain-response model.

### BACKGROUND

The human brain and its underlying biological neural networks have an efficient way to process a small amount of data to promptly reach a cognitive classification. A machine learning (ML) algorithm that can mimic a human brain response may therefore have particular advantages, such as relying on very limited available training to classify content.

For example, in the field of defect inspection of electronic circuits, some inspection activities still have to be performed manually, by human experts. The reason is that there are still human expert capabilities in identifying defects, such as rare defects, or in deciding an outcome of the defect (e.g., disposing of a die due to a given defect), that existing automated inspection techniques cannot provide alternative for.

Another example can be seen in the field of healthcare, where most of the diagnosis processes are performed manually by medical professionals. Although AI models for diagnostics are may become more available, human experts are expected to remain be part of the diagnosis processes, since data harvesting, structuring and labeling, restrict AI from become an autonomous tool in the hand of the medical system.

Accordingly, there is a need for automated visual inspection technique that can replace such human expert reviews.

### SUMMARY OF THE INVENTION

An embodiment of the present invention that is described hereafter provides a content classification method including receiving a set of content items belonging to multiple predefined categories of a specific discipline, and extracting respective features from each content item of the specific discipline. A labeling of the content items of the specific discipline is received, performed by one or more human viewers, the labeling indicating, for each content item, a respective category assigned to the content item by the one or more human viewers from among the multiple predefined categories. A general-content brain-response model is uploaded, the model estimated using measurements of brains of humans presented with a general-content database, wherein the general-content database is defined using a set of features and includes a mapping between the set of features and a set of extracted brain activities. The general-content brain-response model is applied to the extracted features, to calculate, using the labeling, a set of per-category brain-responses for the specific discipline. Given a new content item associated with the discipline, a category that best matches the new content item from among the multiple predefined categories is estimated, based on the general-content brain-response model and the discipline-specific brain responses.

In some embodiments, estimating the category includes (a) extracting a plurality of the features from the new content item, (b) applying the general-content brain-response model to the features extracted from the new content item, to calculate a new content brain-response, and (c) using the set of discipline-specific brain responses and the new content brain-response, estimating the category that best matches the new content item.

In some embodiments, estimating the category includes estimating a respective set of distances, in a brain activity coordinate system, between the new content brain-response and the discipline-specific brain responses, and, using the labelling, classifying the new content item to one of the predefined categories according to the set of distances.

In an embodiment, estimating the category includes calculating a respective set of probabilities that the new content item has a same label as any one of the given categories, and classifying the new content item to one of the predefined categories according to the calculated set of probabilities.

In another embodiment, extracting the features includes omitting from the extracted features one or more of the predefined features that are deemed to be statistically insignificant.

In some embodiments, the extracted features include at least one of shades of colors, characteristic spatial frequencies, contrast levels, and prevalence.

In some embodiments, the content classification method further includes deriving the general-content brain response model using a statistical model that is one of linear regression and non-linear regression.

In an embodiment, the measurements of brains of humans include brain connectivity matrices. In another embodiment, the measurements of brains of humans are modeled based upon cognitive layers combined with a connectivity association matrix.

In some embodiments, the content items of the specific discipline include images of semiconductor dies, wherein the categories are predefined quality bins, and wherein the labeling by the human review includes assignment each image as representing a die belonging to one of predefined quality bins.

In some embodiments, the content classification method further includes deciding on a use of a semiconductor die whose image was classified as representing a die belonging to one of predefined bins.

In an embodiment, the brain measurements are performed by one or more of anatomical Magnetic Resonance Imaging (MRI), Diffusion Tensor Imaging (DTI), Functional MRI (fMRI), Electroencephalogram (EEG), Magnetoencephalogram (MEG), Infrared Imaging, Ultraviolet Imaging, Computed Tomography (CT), Brain Mapping Ultrasound, In-Vivo Cellular Data, In-Vivo Molecular data, genomic data, and optical imaging.

In some embodiments, the labeling includes labeling of at least one of a sequence of frames, images, sounds, tactile signals, odors, tastes, and abstract content type.

In some embodiments, the abstract content type includes feelings.

In an embodiment, the features are represented as a first vector space, wherein the set of brain activities is represented as a second vector space, and wherein the general-content brain-response model is defined as a linear transformation between the first and second vector spaces.

There is additionally provided, in accordance with another embodiment of the present invention, a content classification apparatus including an interface and a processor. The interface is configure to (i) receive a set of content items belonging to multiple predefined categories of a specific discipline, and extracting respective features from each content item of the specific discipline, and (ii) receive a labeling of the content items of the specific discipline, performed by one or more human viewers, the labeling indicating, for each content item, a respective category assigned to the content item by the one or more human viewers from among the multiple predefined categories. The processor is configured to (i) upload a general-content brain-response model estimated using measurements of brains of humans presented with a general-content database, wherein the general-content database is defined using a set of features and includes a mapping between the set of features and a set of extracted brain activities, (ii) apply the general-content brain-response model to the extracted features, to calculate, using the labeling, a set of per-category brain-responses for the specific discipline, and (iii) given a new content item associated with the discipline, estimate a category that best matches the new content item from among the multiple predefined categories, based on the general-content brain-response model and the discipline-specific brain responses.

The present invention will be more fully understood from the following detailed description of the embodiments thereof, taken together with the drawings in which:

### BRIEF DESCRIPTION OF THE DRAWINGS

FIG. 1 is a block diagram schematically illustrating an apparatus that is configured to classify a new content item belonging to a specific discipline using a general brain-response model and a discipline-specific database of labeled brain-responses, according to an embodiment of the present invention;

FIG. 2 is a block diagram, along with a flow chart, that schematically illustrates elements of the apparatus of FIG. 1, and a processing scheme, applied to generate the general brain-response model and use it to derive the database of labeled brain-responses of FIG. 1, according to an embodiment of the present invention;

FIG. 3 is a flow chart that schematically illustrates a method to classify a new content item belonging to a specific discipline using the apparatus of FIG. 1, according to an embodiment of the present invention; and

FIG. 4 is a schematic, pictorial view of a set of estimated distances between a new content item brain response and a scatter of discipline-specific labeled brain-responses, used for classifying a new content item, according to an embodiment of the present invention.

### DETAILED DESCRIPTION OF EMBODIMENTS

### Overview

Embodiments of the present invention that are described hereinafter provide apparatuses and methods for classification of content of a given discipline using a general brain-response model. Examples of disciplines include inspection of semiconductor processing defects, but generally, the disclosed techniques can be used for classification of content pertaining to any other suitable discipline. The content items may comprise, for example, images (e.g., of processed semiconductor dies). Generally, however, the disclosed techniques can be used for classification of other types of content, such as of content items that are audio files.

Typically, the disclosed technique generates and uses a general brain-response model (also called hereinafter “bare brain-response model”) to calculate any required discipline-specific brain-response. The general brain-response model is general, or bare, in the sense that it is not discipline-specific. Generally speaking, the bare brain-response model comprises responses of thousands of brain regions, each region uniquely activated by different attributes of the content. The responses may be estimated, for example, from functional MRI scans of brains of human viewers who are presented with a non-discipline-specific content database, such as a sufficiently large set of images.

The general brain response model covers a very large number of attributes, named hereinafter “features.” The coverage enables simulation of content-specific brain responses, without a need to scan brains of humans exposed to content of the specific discipline to add more content-specific attributes to the model. For example, features such as shades of gray are covered by the general model to an extent that a brain response to a specific content does not require adding a new shade of gray to the feature list of shades of gray.

For a specific classification task, a set of labeled discipline-specific brain-responses is calculated. Given a new content item belonging to the discipline but of unknown label, its brain-response is also calculated.

Using statistical analysis, a category to which the new content belongs (e.g., its label) is estimated by a processor, for example by comparing the mapped response to a scatter of the discipline-specific brain-responses. This way, a new content item of the discipline can be classified. The bare brain-response model, described in detail below, typically comprises one or more mappings (e.g., operators) between a space of features (defining the content items) and a brain-activity space comprising fMRI extracted brain amplitudes.

As indicated above, the disclosed technique relies on the observation that, under certain conditions, a general brain-response model can be built by presenting humans with sufficiently comprehensive general content and scanning their brains in the process. The general brain-response model can subsequently be used, using a database of discipline-specific labeled content items, to calculate respective discipline-specific brain-responses. The discipline-specific brain-responses can later be used for classifying new content of the specific discipline.

For example, a database of general content, such as a set of images that capture a sophisticated urban environment, may be presented to a large number of persons during a brain scan, e.g. fMRI scan, and the fMRI extracted brain responses can then be used to simulate brain responses to discipline-specific images (e.g., of electronic circuits).

The disclosed technique therefore utilizes actual human brains to generate “expert machines” to more closely (e.g., closer than fully artificial models) employ and mimic capabilities of human experts in many different disciplines in order to automatically classify new content. This expert performance is nevertheless achieved by mostly using non-expert humans for generating the bare brain response model, and only a small number of discipline-specific humans (“experts”) for labeling.

Using contents from a complex environment ensures that a sufficiently large set F of features of any content of a same type (e.g., an image) is covered (e.g., sampled) comprehensively enough by the process to serve as a basis for generating different simulated brain-responses that would successfully classify content from respective different disciplines. Indeed, the inventors have found empirically that such a “universal” set F of features can be generated and used to apply the general brain-response model into different successful discipline-specific brain-response classification problems.

A universal set F of such features may comprise, for example, geometries, colors, shades, contrasts, characteristic spatial frequencies obtained using spectral analysis of the image, and prevalence of features, among others. The feature-extraction step typically includes performing initial image preprocessing steps such as smoothing, biasing, and augmenting.

An example of a plurality of categories of a specific discipline are categories of images of processed semiconductor dies. The discipline in such an application is inspection of semiconductor processing defects. In this example, one or more expert human viewers label the contents, and each category includes only images labeled as representing dies of similar (e.g., same bin) quality, with the category labeled accordingly. In other words, different images labeled as showing different die qualities (i.e., dies belonging in different category bins) are each labeled with a different label Lₛ∈G, s=1, 2, . . . M for a set G of M labels. (e.g., each same-labeled group of images corresponds to a single predefined labeled quality bin).

In some embodiments, a preparatory part of the disclosed technique is therefore divided into two phases:

(i) an initial “universal” phase which involves actual brain scans of humans presented with contents of a general-content database. Using a statistical model, a “bare” brain-response model is generated, that establishes a best-fit transformation between a “universal” set of features, F, that defines the general content and a set of brain-response amplitudes (i.e., to F). For example, the set of features can be such that defines a feature space which represents the general content by spanning each feature vector of a content item.

(ii) a discipline-specific phase, during which one or more human experts label specific discipline content items as belonging to one of a group G of predefined categories of the discipline. The bare brain-response model is applied to calculate brain responses to the content items, that are labeled according to category of the labeled content items, thereby obtaining a database of estimated brain-responses of a human expert in that discipline.

In an embodiment, during the initial preparatory phase, a processor extracts, from each of the (digitized) contents of the general-content database, feature set (e.g., feature vector) ƒ belonging to the aforementioned predefined “universal” set of contents (ƒ∈F). For each content item, the respective set ƒ of features ƒ∈F will specify different weights, which can be binary (checked/unchecked) features of the set F (i.e., in some applications some members of ƒ are nulled and are not used).

A bare brain-response model can be constructed from a set of relations (e.g., equations), such as linear relations between features ƒᵢ∈F, described below, and estimated brain amplitudes, {A}, the latter also called “brain-activity maps:”

$${A}_{j} ( {R}_{j} ) = {\sum }_{i = 1}^{n} {α}_{ij} {f}_{i} , 1 \leq j \leq k \qquad Eq . 1$$

where Aⱼ(Rⱼ) is an fMRI extracted activity of brain region Rⱼ, further described below, and where a processor subsequently statistically derives coefficients αᵢⱼ, e.g., by performing linear regression.

In an alternative representation, a vector ƒ of image features, ƒ=(ƒ₁, ƒ₂, . . . , ƒₙ), is extracted for each image shown to a human subject during fMRI scan from which a respective brain activation feature vector, A=(A₁, A₂, . . . , Aₖ), is extracted. A yet unknown mapping Φ(ƒ)∈ [symbol] ^{k} which in Eq. 1 is estimated by a set of linear functions using coefficients αᵢⱼ, can therefore be described herein as one that maps the image features into a higher dimensional (i.e., k>n) brain activations space. The function Φ(ƒ) can be estimated by employing supervised feature learning for example, by modelling Φ(ƒ) by set of k linear functions. That is

Aⱼ=ƒ^{T}βⱼ Eq. 1′

where βⱼ∈ [symbol] ⁿ.

The statistically solved relations of Eq. 1 thereby define a set of “best-fit” coefficients of the relations, denoted hereinafter as array [αᵢⱼ^{F,0}], which can be also described as the aforementioned mapping (e.g., operator) that transforms any vector in a feature space into a respective brain activity vector in a brain activity space.

Note that the structure of Eq. 1 depends on the underlying brain model and statistical model used, as described below. The shown equation is brought by way of example and purely for the sake of simplicity of presentation. For example, a set of non-linear relations may replace Eq. 1.

Thus, more generally, a statistical algorithm, called hereinafter Stat, (e.g., linear or non-linear regression, or a general linear model (GLM), among others) statistically solves a large set of relations (such as the relations of Eq. 1) between the extracted features and the respectively estimated brain amplitudes to each of the images to obtain the general-content brain-response model (or operator):

[αᵢⱼ^{F,0}]=Stat({[αᵢⱼ^{ƒ,0}]}_{ƒ∈F}), Eq. 2

This model, or statistically estimated operator, described by the array of coefficients, [αᵢⱼ^{F,0}], is still “bare,” or implicit, in the sense that the contents (e.g., images) are not application specific, and the set of extracted features therefore do not represent an actual target of classification.

The dimension of the above bare brain-response model is given by example only, whereas the actual dimension may vary according to a complexity (e.g., anatomical, functional) of an underlying brain model used. At the same time, regardless of the exact mathematical description, a sufficiently comprehensive (e.g., large database of) general content ensures that any bare brain-response model used is “full” (e.g., coefficients [αᵢⱼ^{F,0}] are insensitive to enlargement of the database).

As noted above, to construct the aforementioned expert machine (e.g., database of discipline specific brain responses), the processor uploads a discipline-specific set of (digitized) content items that each have respective labels Lₛ∈G. Each labeled category is assumed to include a number Nₛ of content items. The processor extracts from each of the discipline-specific content items a subset F_{ds} of features of the aforementioned universal set of predefined features, F_{ds}⊆F. Subset F_{ds} can be identical to the predefined set F, or be a smaller, optimized subset. For example, in case of a detection, or reception, of an indication of black and white images, the subset of extracted features can be the same set but with zero weights for color shades other than gray (e.g., shades of red). As another example, the elements of ƒ_{ds}, and/or ƒ, can be binary (checked/unchecked) features of set F_{ds} and/or F, respectively. Alternatively, the subset size can be reduced to comprise only shades of gray, i.e. to save memory space and reduce computation effort by not processing nullified shades of other colors.

Either way, for each discipline-specific content, the non-zero elements (i.e., weights) of the respective vector ƒ_{ds} of features ƒ_{ds}∈F_{ds} will vary between content items.

The extraction of feature set F_{ds} involves similar preprocessing steps to the above, and in addition to the aforementioned possible reduction of the universal set of features F into a smaller subset of features, F_{ds}, it also optimizes the model to a specific application (e.g., image classification based on gray scale image contents).

The processor then reapplies the bare brain-response model, [αᵢⱼ^{F,0}] (which specifies brain-response amplitudes in terms of features, ƒᵢ∈F), to calculate a respective set of discipline-specific labeled per category Lₛ∈G brain-responses:

{[αᵢⱼ^{F,0}]ƒ_{ds}}_{ƒ}_{_{ds}}_{∈F}_{_{ds}}_{,L}_{ₛ}_{∈G} Eq. 3

In an embodiment, the set of discipline-specific brain responses given by Eq. 3 can be described as a scatter of vectors in a brain response space.

Given new content item with a yet unknown label, x∈G, that requires classification, the new content item is assumed to belong to one of the plurality of categories of the discipline (e.g., given a new image of a processed semiconductor-die), so the processor extracts from the new content item feature set (e.g., feature vector) ƒ_{nc}∈F_{ds}.

Then the processor applies the brain-response model to the new content item to calculate a new content brain response:

[αᵢⱼ^{F,0}]ƒ_{nc} Eq. 4

where, as noted above, ƒ_{nc}∈F_{ds} is the extracted feature set of the new content. If the new content is a set of different “views” of a same item, such as augmentation set of an image of a semiconductor defect, that generates a set of feature sets ƒ_{nc}∈Fv⊆F_{ds} of the same content (e.g., by augmentation), the new content brain-response can be statistically refined to be:

Stat({[αᵢⱼ^{F,0}]ƒ_{nc}}_{ƒ}_{_{nc}}_{,∈Fv}) Eq. 5

As there are a number Nₛ of discipline specific content items per category, an average of the brain responses of these,

$${\overline{A}}_{s} = \frac{1}{{N}_{s}} {Σ}_{{f}_{ds}}^{{N}_{s}} [ {α}_{ij}^{F , 0} ] {f}_{ds} , \qquad Eq . 6$$

would give an estimate of the label location in a coordinate system of brain activity.

In the alternative representation, the assumed labeled dataset from a specific discipline can be written as {ƒ,Lₛ}ₛ₌₁^{M}, where Lₛ∈G and assuming there are M categories in set of categories G. Note again, that in some applications some members of ƒ are nulled and are not used, with such reduced feature vectors is denoted above as ƒ_{ds}.

The brain activations belonging to a same category (i.e., class) are averaged and the average is denoted by

Ā_{S}=Σ_{S:N}_{ₛ}\hat{Φ}(ƒ_{ds}) Eq. 6′

The prediction of a new feature vector, ƒ_{nc}, is dome based on the similarity between Φ(ƒ_{nc}) and each of the averages Ā_{S}.

In the above description, subsequently, the processor applies a statistical test, such as a similarity test or, in some embodiments, calculates an inner product between the vector [αᵢⱼ^{F,0}]ƒ_{nc} and each of the per label average vectors, to generate a probability of the unknown label x being label Lₛ∈G:

$$P ( x = {L}_{s} ) ∝ \frac{1}{{N}_{s}} {‖ 〈 {Σ}_{{f}_{ds}}^{{N}_{s}} {f}_{ds} [ {α}_{ij}^{F , 0} ] \vert [ {α}_{ij}^{F , 0} ] {f}_{nc} 〉 ‖}^{2} \qquad Eq . 7$$

up to a global normalization factor.

More generally, similarity may be estimated by calculation a distance between the new content brain response [αᵢⱼ^{F,0}]ƒ_{nc} and the labeled scatter {[αᵢⱼ^{F,0}]ƒ_{ds}}_{ƒ}_{_{ds}}_{∈F}_{_{ds}}_{,L}_{ₛ}_{∈G}. An L₁ or other distances may be used for estimating similarity, such as the Hellinger distance.

In case an inner product is used, using the labels, the processor classifies the content according to the probability distribution and classification criteria applied to the distribution. For example, the processor may select the most likely label as the result of the classification. More generally, the processor classifies the content according to a level of similarity between the mapped new content and mapped labeled contents, as further described below.

In some embodiments, the processor further outputs a decision based on the classification. For example, using prespecified acceptance criteria, the processor may issue a decision to reject a semiconductor die based on the classification, and to direct the die to in-house root cause analysis.

To classify contents from very different disciplines, including non-visual ones such as feelings, tones of speech, etc., the step of content preprocessing may include transforming content between content types for further analysis using the disclosed technique. For example, the preprocessing may include encoding audio files into images.

Alternatively, contents belonging to any non-visual category may have their own type of database of defined relevant features, such as, for audio, loudness level and characteristic sound frequencies. The inventors have found, however, that a processor can be programmed to perform different preprocessing steps instead, e.g., to encode audio information into visual information and use extracted features of a visual content in a verifiable manner.

Similarly, other non-visual content types, such as smells and abstract content types (e.g., feelings), may be successfully encoded into a multi-dimensional visual scale and classified using the disclosed technique.

### APPARATUS DESCRIPTION

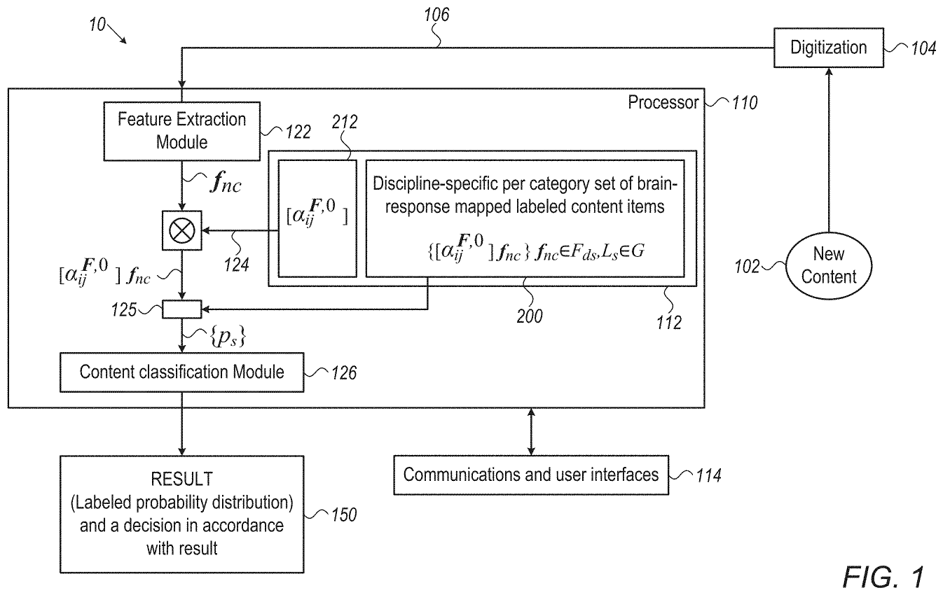

FIG. 1 is a block diagram schematically illustrating an apparatus 10 that is configured to classify a new content item 102 belonging to a specific discipline using a general brain-response model 212 and a discipline-specific database 200 of labeled brain-responses, according to an embodiment of the present invention. Discipline-specific new content item 102 can be, for example, an image of a processed semiconductor die.

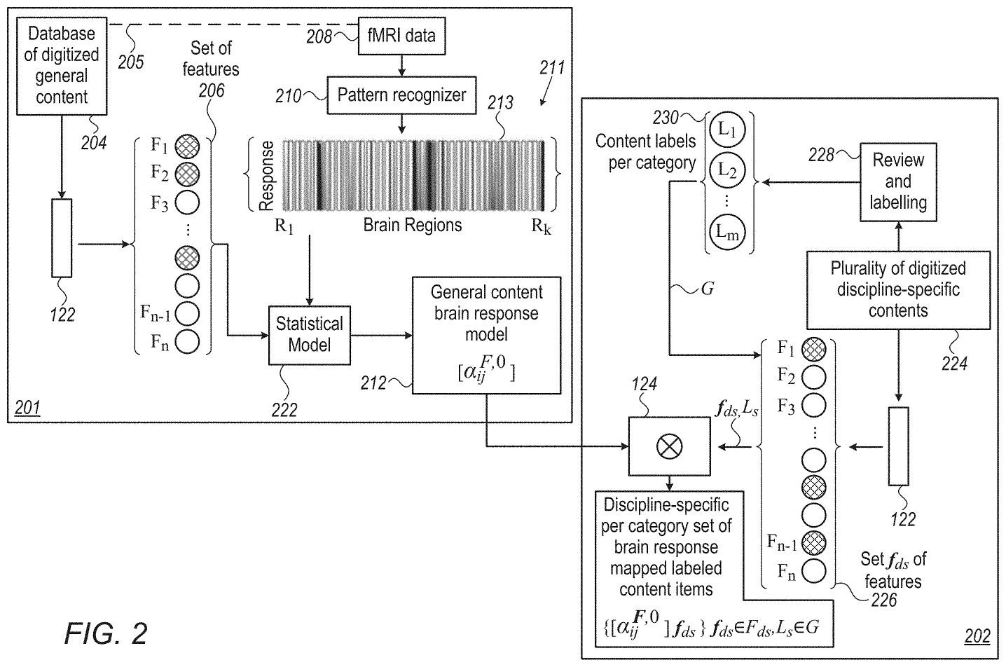

For apparatus 10 to perform its intended use, a memory 112 of apparatus 10 holds database 200, comprising a set, per category of previously calculated, as described in FIG. 2, labeled brain-responses {[αᵢⱼ^{F,0}]ƒ_{ds}}_{ƒ}_{_{ds}}_{∈F}_{_{ds}}_{,L}_{ₛ}_{∈G}. In other words, processor 110 holds in memory 112 an “expert-machine” for the discipline.

Digitization module 104 digitizes the new content 102 into a digital data set, called hereinafter a digital image, for input to apparatus 10. The digital image is communicated (106) (e.g., via the network, or via a local communication link) to a processor 110 of apparatus 10. The digital image may be stored in memory 112.

A feature extraction module 122 of processor 110 runs a feature recognition algorithm to extract a respective new content feature set (e.g., feature vector) ƒ_{nc} from new content 102. Typically, feature extraction module 122 may include one or more of the aforementioned preprocessing steps, suitable for use with contents of the specific discipline, to assist in extracting feature vector ƒ_{nc}.

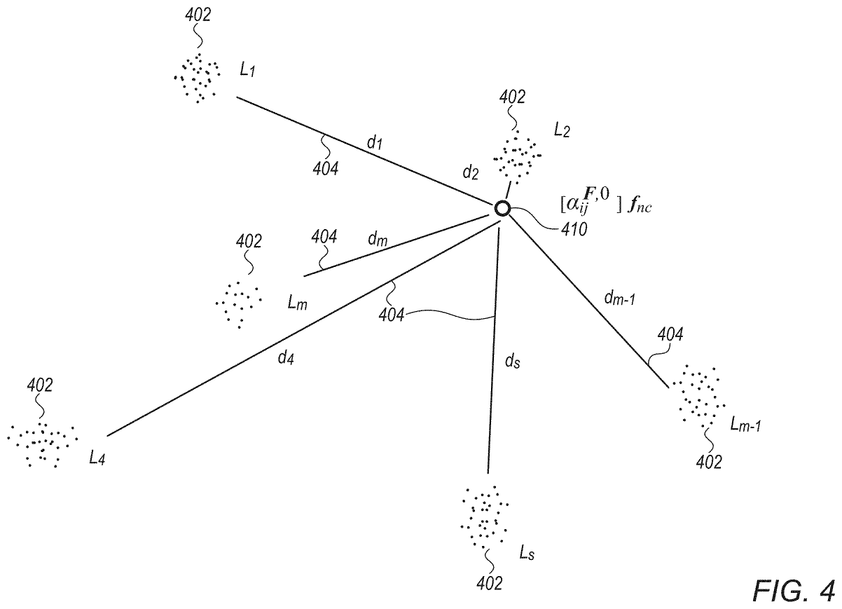

Processor 110 inputs feature vector ƒ_{nc} into multiplication module 124, which derives a brain-response model for the new content, [αᵢⱼ^{F,0}]ƒ_{nc}. At this stage, the new content has the aforementioned, as yet unknown, label x∈G. Upon receiving an input, a statistical test module 125 estimates mapped brain response data point (e.g., vector) [αᵢⱼ^{F,0}] ƒ_{nc} relative to the labeled mapped brain responses {[αᵢⱼ^{F,0}]ƒ_{ds}}_{ƒ}_{_{ds}}_{∈F}_{_{ds}}_{,L}_{ₛ}_{∈G}, all of which can be vectors in a brain activity space, or more generally, as shown in FIG. 4, data points in a brain activity coordinate system. Subsequently, statistical test module 125 runs a similarity test to calculate by, for example, calculating using Eq. 7, an inner product of the vectors, a probability p=pₛ that x=Lₛ that a human expert would have classified and labeled new content 102 as belonging to category L_{S}. More generally the two sets undergo a similarity check, as described below, with a set of distances estimated, as seen in FIG. 4.

To cover all labels, module 125 calls (temporal logic not shown) the entire stored set 200 of discipline-specific set of per category Lₛ∈G brain-responses, to generate a probability distribution of new content item 102 belonging to one of the labeled sets of the discipline-specific contents.

A content classifier 126 arranges a result 150 comprising the labeled and optionally graded probability distribution. To this end classifier 126 may apply predefined criteria, such as selecting the maximal probability label, a minimal distance. Classifier 126 outputs a decision in accordance with the result, for example to a user display (not shown).

For example, in case of an image of a processed semiconductor die, the result may be limited by module 126 to three options, 97% probability that the die should be labeled as belonging to a “good” bin, 2% probability that the die should be labeled as belonging to an “acceptable” bin, and 15 probability that the die should be labeled as belonging to a “rejected” bin. The decision may be based, for example, on criteria stored in memory 112 having a criterion of 0.1% as the maximum probability allowed for a “rejected” bin, to reject the die based on the above classification, and to transfer the die to human inspection for root cause analysis.

Processor 110 may be engaged locally or remotely, using communications and user interfaces 114, such as query from a remote program/user or a selection/query by a user using, for example, a touchscreen.

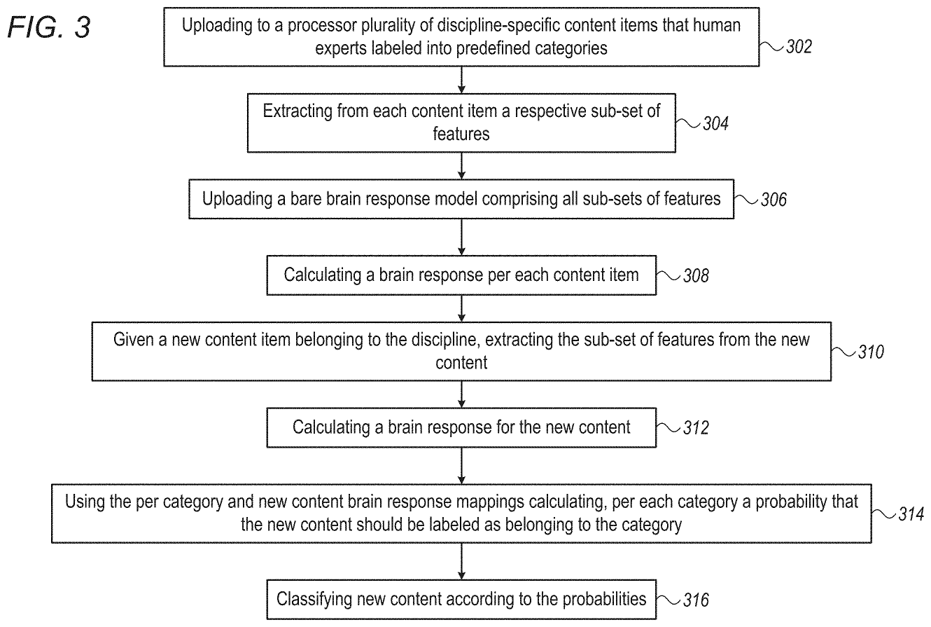

In various embodiments, the different electronic elements of the apparatus shown in FIG. 1 may be implemented using suitable hardware, such as using one or more discrete components, one or more Application-Specific Integrated Circuits (ASICs) and/or one or more Field-Programmable Gate Arrays (FPGAs). Some of the functions of the disclosed processors, units, and modules, e.g., some or all functions of processor 110 and its modules 122-126, may be implemented in one or more general purpose processors, which are programmed in software to carry out the functions described herein. The software may be downloaded to the processors in electronic form, over a network or from a host, for example, or it may, alternatively or additionally, be provided and/or stored on non-transitory tangible media, such as magnetic, optical, or electronic memory. In particular, processor 110 runs a dedicated algorithm as disclosed herein, including in FIG. 3, that enables processor 110 to perform the disclosed steps, as further described below.

### Generation of a Content-Specific Labeled Set of Brain-Responses

FIG. 2 is a block diagram, along with a flow chart, that schematically illustrates elements (122, 124) of apparatus 10 of FIG. 1, and a processing scheme, applied to generate the general brain-response model 212 and use it to derive database 200 of labeled brain-responses of FIG. 1, according to an embodiment of the present invention.

The shown processing is divided into two phases:

(i) phase 201 which involves actual brain scans to generate, using a statistical model 222, the general-content brain-response model [αᵢⱼ^{F,0}] (212), that connects features of a general-content database 204 and a respective set 211 of fMRI extracted brain amplitudes, and

(ii) phase 202, in which general-content brain-response model 212 is applied to calculate the discipline-specific set of per category L_{S}∈G brain-responses {[αᵢⱼ^{F,0}]ƒ_{ds}}_{ƒ}_{_{ds}}_{∈F}_{_{ds}}_{,L}_{ₛ}_{∈G}, for each labeled set, or vector, ƒ_{ds} 226 of features of the discipline-specific contents (e.g., for each labeled images of semiconductor dies) of database 224. In this way, predictive or classifying powers of human brains are utilized to model expert machines in multiple disciplines.

The generation of database 200 includes an algorithm, according to the presented embodiment, that is used by apparatus 10 to carry out a process that begins with receiving a database 204 of digitized contents, such as a set of color images that captures a sophisticated urban environment, and/or captures another sophisticated environment, such as various human activities and gestures, to give two examples.

Using module 122 of FIG. 1, processor 110 extracts features (black-filled circles) belonging to a predefined set 206 of features F=(ƒ₁, ƒ₂, . . . , ƒₖ) from each content (i.e., digitized image) of the general-content database 204. Such extraction typically includes preliminary steps of preprocessing and pattern recognition.

As indicated above, some of the features of the vector (ƒ₁, ƒ₂, . . . , ƒₖ) are checked, per content, and some are not, to produce feature vector ƒ_{content} that a content a specific item represents the content. For example, color features are unchecked in a gray scale image, and some geometric features may be checked or unchecked, such as arcs missing in an image of an object that includes only straight lines and straight angles.

Processor 110 further receives a respective database of fMRI data (208) comprising fMRI images of human brains of people presented with the content of database 204. While data 208 is presented as fMRI data, it can be of other types, such as electroencephalograms (EEG), magnetoencephalograms (MEG), infrared images, ultraviolet images, computed tomography (CT), ultrasound images, in-vivo cellular data, in-vivo molecular data, genomic data, or optical imaging.

Using a prespecified brain model, such as a map of brain regions and/or of neural paths of the brain that may be activated, a pattern recognition module 210 generates from brain measurements (e.g., fills in the model with such data) a respective set 211 of brain-response amplitudes 213 per image presented to the human, response 213 comprising, for example, brain activity amplitudes Aⱼ(Rⱼ) of Eq. 1.

Mathematically, modeling activation of certain brain regions and/or neural paths (the latter also called “brain connectome”) by medical imaging and other measurement tools can be realized by substituting a set of matrices of a general human brain-response model, fMRI extracted response values correlative to features of the presented content to generate brain activity maps, thereby providing the set amplitudes Aⱼ(Rⱼ) of aforementioned general-content brain-response model (operator).

In the shown embodiment, set 211 of fMRI extracted brain amplitudes (e.g., a set of brain activity maps 213) is shown to comprise a gray level value of response per brain region (R₁, R₂, . . . , Rₖ). The brain activity amplitudes (e.g., the gray shade barcodes) may represent the level of activation using, for example, an m-bit scale giving a numeric index that represents the amplitude of brain activity within each brain region Rⱼ, while taking into consideration (in another array, not shown) the interaction (e.g., coupling) between the brain regions (i.e., weighted according to the connectome and represented, for example, as off diagonal elements of the set of matrices of a general human brain-response model in which the diagonal elements stand for brain region values).

Statistical model 222 therefore statistically derives (e.g., using multidimensional regression) a best fit bare brain-response model 212 (i.e., best fit operator [αᵢⱼ^{F,0}]) to transform a vector of extracted features ƒ_{content} to a respective array of brain-response amplitudes 213, and stores the operator in memory, for use in generating, using Eqs. 1 and 2, general-content brain-response model [αᵢⱼ^{F,0}](212).

In phase 202, one or more expert humans review and label (228) a set 224 of discipline-specific contents. For example, if the contents are images of processed semiconductor dies, such experts can be defect inspection engineers. The experts can select any label from a predefined group of possible labels, G=(L₁, L₂, . . . , Lₘ). The predefined group of labels may include, for sake of example, labels such as L₁=“highest bin” (e.g., totally clean die),” L₂=“high bin,” L₃=“medium bin,” L₄=“low bin,” L₅=“lowest bin,” and L₆=“rejected,” where, in general, the result of the reviewing and labeling process 228 should cover all labels of set 230 G=(L₁, L₂, . . . , Lₘ).

In parallel, processor 110 extracts discipline-specific contents from set 224 using feature extraction module 122, a respective subset 226 of discipline-specific features.

At this step the estimated bare model 212 is an applied discipline-specific subset of features 226, and using calculates per each label Lₛ∈G, the discipline-specific per labeled category L_{S} brain-responses {[αᵢⱼ^{F,0}]ƒ_{ds}}_{ƒ}_{_{ds}}_{∈F}_{_{ds}}_{,L}_{ₛ}_{∈G}.

Returning to FIG. 1, given the new content item of the discipline that is yet to be classified, e.g., still with the as yet unknown label x∈G, and having a feature set ƒ_{nc}, general-content brain-response model 212 is applied to calculate the brain response for the new content, using Eq. 4. Statistical test module 125 then outputs a probability per category of label x being one of labels Lₛ∈G, as described above.

Result 150 of FIG. 1 can thus be a probability distribution P=(p₁, p₂, . . . , pₘ) of the label of the new content (e.g., new image from the discipline) being one of the labels (L₁, L₂, . . . , Lₘ), or, p=pₛ that x=Lₛ. Such probabilities can be calculated, for example, using Eq. 7.

In the case of the semiconductor die example, result 150 of FIG. 1 may be a probability distribution (p₁, p₂, . . . , p₆) of the label of the new image being one of the above listed labels (L₁, L₂, . . . , L₆).

Subsequently, for example, based on acceptance criteria, such as thresholding of probability amplitudes, or levels of incidence per label, the probability distribution is converted into a decision-making process (e.g., a commercial decision, such as determining that the imaged die is suitable for a high-end use, for a low-end use, or should be discarded.

### Method of Classifying New Content

FIG. 3 is a flow chart that schematically illustrates a method to classify a new content item belonging to a specific discipline using apparatus 10 of FIG. 1, according to an embodiment of the present invention. The algorithm, according to the present embodiment, carries out a process that uses general-content brain-response model 212 and discipline-specific per category labeled brain-responses 200 stored in a memory 112 of apparatus 10. An example of new content is the aforementioned image of a processed semiconductor die taken by an inspection apparatus to detect semiconductor processing defects.

The process begins with a discipline-specific contents uploading step 302, in which processor 110 uploads a plurality of content items labeled as different categories Lₛ∈G, of a specific discipline, such as semiconductor processing defect inspection, where each category has a different label Lₛ that ranges from labels of benign defects to labels of critical defects. In particular, one of the categories of contents may be of reference images of a “golden-die,” i.e., images of a defect-free die). The contents are assumed to be already in a digital format.

Next, at a discipline-specific feature extraction step 304, feature extraction module 122 of processor 110 performs preprocessing and extracts a respective set of feature vectors ƒ_{ds}∈F_{ds} and stores them in memory 112.

Next, at a bare brain-model response uploading step 306, processor 110 uploads from memory 112, bare brain-response model 212, [αᵢⱼ^{F,0}].

Next, at a discipline-specific brain-response calculating step 308, using set of labeled (230) feature vectors ƒ_{ds}∈F_{ds}, bare model 212 is applied by processor 110 to calculate brain-responses {[αᵢⱼ^{F,0}]ƒ_{ds}}_{ƒ}_{_{ds}}_{∈F}_{_{ds}}_{,L}_{ₛ}_{∈G}, i.e., in the aforementioned expert-machine database 200 for the discipline.

At a new content feature extraction step 310, given a new content item with a yet unknown label, x∈G (e.g., given a new image of a processed semiconductor-die), processor 110 extracts a feature vector ƒ_{nc}∈F_{ds} from the new content item.

At a new content mapping step 312, calculate the brain response for the new content, using Eq. 4., [αᵢⱼ^{F,0}]ƒ_{nc}.

At a labeling step 314, using a statistical test comprising, for example, Eq. 7, processor 110 generates a probability p=pₛ that X=Lₛ. Specifically, with Eq. 7, processor 110 calculates the component of the vector [αᵢⱼ^{F,0}]ƒ_{nc} at a direction [αᵢⱼ^{F,0}]ƒ_{ds} in a brain activity space, using module 125.

Finally, at a classification step 316, module 126 of processor 110 classifies (e.g., selects a most probable label for, or a best match to) the new content item, based on the probability distribution P and other prespecified classification criteria, such as a set of the aforementioned distances, shown in FIG. 4.

The flow chart of FIG. 3 is brought by example, purely for the sake of clarity. While the inspection process described by FIG. 3 is exemplified for wafer dies, other electronic circuits, such as circuitries of a PCB, may be inspected in a similar way. While the example given in FIG. 3 of the disclosed method is from a specific discipline, the method can, mutatis mutandis, be constructed and applied to numerous disciplines, ranging from engineering to psychology, and from law enforcement to entertainment.

FIG. 4 is a schematic, pictorial view of a set of estimated distances 404 between a new content item brain response 410 and a scatter of discipline-specific labeled brain-responses 402, used for classifying a new content item, according to an embodiment of the present invention.

As seen, new content data point 410 has a different distance (e.g., an L₁ distance) from the labeled sets 402 of brain responses, with distance d₂ from L₂ labeled set 402 being the minimal of these and distance d₄ from L₄ labeled set 402.

In one embodiment, processor 110 labels the new content with label L₂. In another embodiment, processor 110 generates a probability distribution based on the inverse of the distances d₁, d₂, . . . , dₛ, . . . , dₘ, and labels data point 410 with labels L₁, L₂, . . . , Lₛ, . . . , Lₘ with a probability according to the distance-derived probability distribution.

The view of FIG. 4 is brought by example, purely for the sake of clarity. Additional steps may be performed, which are omitted for simplicity. For example, in an embodiment, each of the scatter 402 is initially averaged into a single data point, and the distances are calculated between mean values. The example given in FIG. 4 can be constructed and applied in numerous disciplines.

It will be thus appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art.

1. A content classification method, comprising:
receiving a set of content items belonging to multiple predefined categories of a specific discipline, and extracting respective features from each content item of the specific discipline;
receiving a labeling of the content items of the specific discipline, performed by one or more human viewers, the labeling indicating, for each content item, a respective category assigned to the content item by the one or more human viewers from among the multiple predefined categories;
uploading a general-content brain-response model estimated using measurements of brains of humans presented with a general-content database, wherein the general-content database is defined using a set of features and comprises a mapping between the set of features and a set of extracted brain activities;
applying the general-content brain-response model to the extracted features, to calculate, using the labeling, a set of per-category brain-responses for the specific discipline; and
given a new content item associated with the discipline, estimating a category that best matches the new content item from among the multiple predefined categories, based on the general-content brain-response model and the discipline-specific brain responses.

2. The content classification method according to claim 1, wherein estimating the category comprises:
extracting a plurality of the features from the new content item;
applying the general-content brain-response model to the features extracted from the new content item, to calculate a new content brain-response; and
using the set of discipline-specific brain responses and the new content brain-response, estimating the category that best matches the new content item.

3. The content classification method according to claim 2, wherein estimating the category comprises estimating a respective set of distances, in a brain activity coordinate system, between the new content brain-response and the discipline-specific brain responses, and, using the labelling, classifying the new content item to one of the predefined categories according to the set of distances.

4. The content classification method according to claim 1, wherein estimating the category comprises calculating a respective set of probabilities that the new content item has a same label as any one of the given categories, and classifying the new content item to one of the predefined categories according to the calculated set of probabilities.

5. The content classification method according to claim 1, wherein extracting the features comprises omitting from the extracted features one or more of the predefined features that are deemed to be statistically insignificant.

6. The content classification method according to claim 1, wherein the extracted features comprise at least one of shades of colors, characteristic spatial frequencies, contrast levels, and prevalence.

7. The content classification method according to claim 1, and comprising deriving the general-content brain response model using a statistical model that is one of linear regression and non-linear regression.

8. The content classification method according to claim 1, wherein the measurements of brains of humans comprise brain connectivity matrices.

9. The content classification method according to claim 8, wherein the brain connectivity matrices include connectivity-matrix weights based upon micro-structure estimates of brain tissue to form a brain connectome.

10. The content classification method according to claim 1, wherein the measurements of brains of humans are modeled based upon cognitive layers combined with a connectivity association matrix.

11. The content classification method according to claim 1, wherein the content items of the specific discipline comprise images of semiconductor dies, wherein the categories are predefined quality bins, and wherein the labeling by the human review comprises assignment each image as representing a die belonging to one of predefined quality bins.

12. The content classification method according to claim 11, and comprising deciding on a use of a semiconductor die whose image was classified as representing a die belonging to one of predefined bins.

13. The content classification method according to 1, wherein the brain measurements are performed by one or more of anatomical Magnetic Resonance Imaging (MRI), Diffusion Tensor Imaging (DTI), Functional MRI (fMRI), Electroencephalogram (EEG), Magnetoencephalogram (MEG), Infrared Imaging, Ultraviolet Imaging, Computed Tomography (CT), Brain Mapping Ultrasound, In-Vivo Cellular Data, In-Vivo Molecular data, genomic data, and optical imaging.

14. The content classification method according to claim 1, wherein the labeling comprises labeling of at least one of a sequence of frames, images, sounds, tactile signals, odors, tastes, and abstract content type.

15. The content classification method according to claim 14, wherein the abstract content type comprises feelings.

16. The content classification method according to claim 1, wherein the features are represented as a first space, wherein the set of brain activities is represented as a second space, and wherein the general-content brain-response model is defined as a linear transformation between the first and second spaces.

17. A content classification apparatus, comprising:
an interface, which is configured to:
receive a set of content items belonging to multiple predefined categories of a specific discipline, and extracting respective features from each content item of the specific discipline; and
receive a labeling of the content items of the specific discipline, performed by one or more human viewers, the labeling indicating, for each content item, a respective category assigned to the content item by the one or more human viewers from among the multiple predefined categories; and
a processor, which is configured to:
upload a general-content brain-response model estimated using measurements of brains of humans presented with a general-content database, wherein the general-content database is defined using a set of features and comprises a mapping between the set of features and a set of extracted brain activities;
apply the general-content brain-response model to the extracted features, to calculate, using the labeling, a set of per-category brain-responses for the specific discipline; and
given a new content item associated with the discipline, estimate a category that best matches the new content item from among the multiple predefined categories, based on the general-content brain-response model and the discipline-specific brain responses.

18. The content classification apparatus according to claim 1, wherein the processor is configured to estimate the category by:
extracting a plurality of the features from the new content item;
applying the general-content brain-response model to the features extracted from the new content item, to calculate a new content brain-response; and
using the set of discipline-specific brain responses and the new content brain-response, estimating the category that best matches the new content item.

19. The content classification apparatus according to claim 18, wherein the processor is configured to estimate the category by estimating a respective set of distances, in a brain activity coordinate system, between the new content brain-response and the discipline-specific brain responses, and, using the labelling, classifying the new content item to one of the predefined categories according to the set of distances.

20. The content classification apparatus according to claim 17, wherein the processor is configured to estimate the category by calculating a respective set of probabilities that the new content item has a same label as any one of the given categories, and classifying the new content item to one of the predefined categories according to the calculated set of probabilities.

21. The content classification apparatus according to claim 17, wherein the processor is configured to extract the features by omitting from the extracted features one or more of the predefined features that are deemed to be statistically insignificant.

22. The content classification method according to claim 17, wherein the extracted features comprise at least one of shades of colors, characteristic spatial frequencies, contrast levels, and prevalence.

23. The content classification apparatus according to claim 17, wherein the processor is further configured to derive the general-content brain response model using a statistical model that is one of linear regression and non-linear regression.

24. The content classification apparatus according to claim 17, wherein the measurements of brains of humans comprise brain connectivity matrices.

25. The content classification apparatus according to claim 24, wherein the brain connectivity matrices include connectivity-matrix weights based upon micro-structure estimates of brain tissue to form a brain connectome.

26. The content classification apparatus according to claim 17, wherein the measurements of brains of humans are modeled based upon cognitive layers combined with a connectivity association matrix.

27. The content classification apparatus according to claim 17, wherein the content items of the specific discipline comprise images of semiconductor dies, wherein the categories are predefined quality bins, and wherein the labeling by the human review comprises assignment each image as representing a die belonging to one of predefined quality bins.

28. The content classification apparatus according to claim 27, wherein the processor is further configured to deciding on a use of a semiconductor die whose image was classified as representing a die belonging to one of predefined bins.

29. The content classification apparatus according to claim 17, wherein the brain measurements are performed by one or more of anatomical Magnetic Resonance Imaging (MRI), Diffusion Tensor Imaging (DTI), Functional MRI (fMRI), Electroencephalogram (EEG), Magnetoencephalogram (MEG), Infrared Imaging, Ultraviolet Imaging, Computed Tomography (CT), Brain Mapping Ultrasound, In-Vivo Cellular Data, In-Vivo Molecular data, genomic data, and optical imaging.

30. The content classification apparatus according to claim 17, wherein the labeling comprises labeling of at least one of a sequence of frames, images, sounds, tactile signals, odors, tastes, and abstract content type.

31. The content classification apparatus according to claim 30, wherein the abstract content type comprises feelings.

32. The content classification apparatus according to claim 17, wherein the features are represented as a first space, wherein the set of brain activities is represented as a second space, and wherein the general-content brain-response model is defined as a linear transformation between the first and second spaces.
